SEQUENCE LISTING (4 sequences)

SEQ ID NO: 1 - protein, 20 aa, Homo sapiens
SPRCHLGPGHQAGPGLHRPP

SEQ ID NO: 2 - protein, 6 aa, Homo sapiens
APDTRP

SEQ ID NO: 3 - protein, 13 aa, Homo sapiens
GVTSAPDTRPAPG

SEQ ID NO: 4 - protein, 9 aa, Homo sapiens
LSFYSGAQR

(19) United States
(12) Patent Application Publication
(10) Pub. No.: US 2025/0110120 A1
(43) Pub. Date: Apr. 3, 2025

(54) METHOD FOR DETERMINATION OF MUTATED FORM OF MUCIN-1 PROTEIN IN A BIOLOGICAL SAMPLE

(71) Applicants: GENESPECTOR S.R.O., Praha (CZ); WAKE FOREST UNIVERSITY HEALTH SCIENCES, Winston-Salem, NC (US)

(72) Inventors: Stanislav KMOCH, Praha (CZ); Martina ZIVNA, Praha (CZ); Jan ZIVNY, Praha (CZ); Helena TRESLOVA, Praha (CZ); Anthony BLEYER, Winston-Salem, NC (US); Kendrah KIDD, Winston-Salem, NC (US)

(21) Appl. No.: 18/833,026

(22) PCT Filed: Jan. 31, 2022
(86) PCT No.: PCT/CZ2022/050009

§ 371 Date: Jul. 25, 2024

(30) Foreign Application Priority Data
Jan. 25, 2022 (CZ) PV 2022-34

(51) Int. Cl. G01N 33/543; G01N 33/68

(52) U.S. Cl. CPC G01N 33/543; G01N 33/6854; G01N 33/6893; G01N 2333/4725; G01N 2800/347; G01N 2800/52

(57) ABSTRACT

Method for determination of mutated form of mucin-1 protein in a biological sample comprising steps of adding biological sample to immobilized antibody against mutated form of VNTR MUC1fs, adding antibody against wild-type VNTR MUC1fs to portion of this mixture and performing quantitative analysis, wherein antibody against mutated form of VNTR MUC1fs is added to remainder of mixture of biological sample with immobilized antibody against mutated form of VNTR MUC1fs quantitative analysis is performed. Determination of concentration of MUC1fs in sample and determination of length of mutated or wild-type VNTR MUC1fs in sample is performed using appropriate calibration curves obtained by analyzing standard containing at least one epitope of mutated and at least one epitope of wild-type VNTR MUC1fs.

### TECHNICAL FIELD

The invention relates to immunoassays and biological material analysis associated with a determination of proteins contained therein using a biospecific ligand binding methods comprising an insoluble support for immobilizing immunochemicals and including monoclonal antibodies.

### BACKGROUND ART

Autosomal Dominant Tubulointerstitial Kidney Disease (ADTKD) is the second most common group of genetic kidney diseases after polycystosis. ADTKD is characterized by a dominant inheritance, normal urine and blood laboratory tests (except elevated serum creatinine and in some cases uric acid), tubular atrophy, interstitial fibrosis, and progressive renal failure requiring dialysis and transplantation. One category of ADTKD is a group associated with mutations in the MUC1 gene (ADTKD-MUC1, formerly known as MCKD1). To date, all known cases of ADTKD-MUC1 are caused by frameshift mutations, i.e. insertions or deletions, in the MUC1 gene, which is highly polymorphic due to the presence of variable number tandem repeats (VNTR), whereas in the MUC1 gene, there can be 20-125 repetitions each consisting of 60±3n base pairs, and also due to the occurrence of single nucleotide polymorphisms that affect MUC1 mRNA splicing. Due to alternative splicing, MUC1 pre-mRNA encodes several different proteins, including mucin-1 (MUC1). MUC1 is expressed in epithelial cells of a mucosal surface of several organs, especially the kidneys, lungs, breasts, intestines, esophagus, salivary glands, and pancreas. A key characteristic of MUC1 is a multiple O-glycosylation of the VNTR region, which plays a role as a signaling structure and gives this protein slimy properties.

The above-mentioned mutations in the form of insertions or deletions of DNA segments of different lengths lead to a frameshift during translation and, as a result, to the synthesis of a mutated variant of MUC1 (MUC1fs, frame-shifted). In a majority of the population affected by ADTKD-MUC1, this mutation occurs mostly within the VNTR region and only rarely before this region. Thus, in most cases, MUC1fs contains a partly natural, so-called wild-type, VNTR region at the N-terminus, i.e. the region encoded by the pre-mutated portion of the gene, and a mutated form of the VNTR region at the C-terminus, i.e. behind the mutation. The mutated form of the VNTR region contains 80% fewer serine and threonine amino acids, which are normally extensively glycosylated, and is characterized by the presence of cysteine, leucine, histidine, and arginine amino acids. This sequence change determines that MUC1fs is an abnormal, non-naturally occurring, cysteine-rich, and highly basic protein that has completely unique biophysical properties, for example an isoelectric point >12. At the same time, due to the altered amino acid sequence, MUC1fs does not contain the usual structures at the C-terminus important for posttranslational modi-fications, cell signaling, proper anchoring in the cell membrane, and other molecular-biology events inherent to MUC1. As a result of these changes, MUC1fs accumulates within MUC1-producing cells and, in the case of the kidneys, causes their progressive failure. In patients diagnosed with ADTKD-MUC1, accumulation of MUC1fs has also been demonstrated in other tissues in which MUC1 is synthesized, such as breast tissue, lung epithelium, cutaneous sebaceous glands, or gastrointestinal mucosa, however, for unknown reasons, it is not toxic to these tissues and is without any clinical manifestation.

ADTKD-MUC1 therapy is currently limited to symptomatic treatment, such as supporting kidney function, dialysis, or kidney transplantation. Causal treatment is in an experimental stage, while three possible approaches are studied. First approach is a targeted blockade of the MUC1 promoter, which prevents the synthesis of mutated MUC1fs, second approach is an application of a pharmaceutically active substance allowing MUC1fs to permeate into lysosomes where it degrades, where it degrades, thereby reducing the accumulated amount, and third approach is an application of a substance that increases the permeability of MUC1fs through the cell membrane into the extracellular space and its release it into the circulation, which again leads to a reduction of the amount accumulated inside cells, and thus relief to the kidney tissue.

Due to the high variability caused by the different length of the VNTR region and the possibility of the mutation itself being anywhere within this region, it is impossible to detect ADTKD-MUC1 in a wide range of variants by standard genetic testing methods, and therefore it is very difficult to diagnose. Genetic, spectrometry-based assays (Blumenstiel B, DeFelice M, Birsoy O, et al. Development and Validation of a Mass Spectrometry-Based Assay for the Molecular Diagnosis of Mucin-1 Kidney Disease. J Mol Diagn 18:566-571, 2016) or amplicon sequencing (Wenzel A, Altmueller J, Ekici A B, et al. Single molecule real time sequencing in ADTKD-MUC1 allows complete assembly of the VNTR and exact positioning of causative mutations. Sci Rep 8:4170, 2018; Wang G Q, Rui H L, Dong H R, et al., SMRT sequencing revealed to be an effective method for ADTKD-MUC1 diagnosis through follow-up analysis of a Chinese family. Sci Rep 10, 2020) are known from prior art. However, these methods require complicated instrumental and can detect only a few specific variants of genetic mutations associated with ADTKD-MUC1. Furthermore, methods using immunohistochemical staining to detect the presence of MUC1fs in urinary cell swabs or in tissues obtained by kidney biopsies are known (Zivna M, Kidd K, Pristoupilova A, et al. Non-invasive Immunohistochemical Diagnosis and Novel MUC1 Mutations Causing Autosomal Dominant Tubulointerstitial Kidney Disease. J Am Soc Nephrol 29:2418-2431, 2018; Knaup K X, Hackenbeck T, Popp B, et al., Biallelic Expression of Mucin-1 in Autosomal Dominant Tubulointerstitial Kidney Disease: Implications for Nongenetic Disease Recognition. J Am Soc Nephrol 29:2298-2309, 2018). However, these methods are associated with highly invasive sampling and, above all, do not allow the determination of free MUC1fs in blood plasma.

There are currently no known techniques for a quantitative determination of free MUC1fs in blood plasma. Such an assay would provide a number of advantages in the context of clinical practice, as it would enable easy, fast, and minimally invasive differential diagnosis of hereditary chronic kidney disease as well as monitoring of a progression of this disease and an effectiveness of causal therapy by determining plasma levels of MUC1fs. It would also find an application in cancer diagnosis and therapy. In some types of tumors, there is a somatic mutation in the MUC1 gene, due to which the tumor cells produce MUC1fs. Therefore, it is a suitable biomarker for diagnosing and monitoring the progression and the effectiveness of tumor therapy. Genetic tests, which are currently a standard in the field of ADTKD-MUC1 diagnosis, although very limited in terms of effectiveness, are not capable of any of the abovementioned monitoring of progression or therapy, simply based on their principle of action. Only an indirect method of diagnosing ADTKD-MUC1 by determination of non-mutated MUC1 in blood plasma is known from prior art, as in some patients suffering from ADTKD-MUC1, natural levels of non-mutated MUC1 are reduced in comparison with healthy population (Vyletal P, Kidd K, Ainsworth H C, Plasma Mucin-1 (CA15-3) Levels in Autosomal Dominant Tubulointerstitial Kidney Disease due to MUC1 Mutations Am J Nephrol 1-10, 2021). However, based on its principle, this method also cannot be used to monitor the progression of the disease, whether ADTKD-MUC1 or cancer, or the effectiveness of a therapy. Quantitative determination of free MUC1fs in blood plasma is further complicated by the highly variable length of the VNTR region, which varies significantly in the population, and the different location of the mutation in individual patients and their relatives. Thus, high demands are placed on determination techniques, such as the need to detect and determine an almost unlimited number of variants.

### SUMMARY OF INVENTION

The present invention is based on a system comprising an antibody against a mutated form of VNTR region immobilized on a solid support that binds a mutated MUC1fs protein and antibodies against both wild-type VNTR (wt-VNTR) and mutated VNTR (fs-VNTR, frame-shifted) containing a group that enables quantitative detection. By comparing both normalized obtained signals, it is possible to determine, among other things, the position of the mutation and the relative length of individual forms of VNTR. The invention presents a method of determination carried out on this system. The present invention overcomes the aforementioned shortcomings of the prior art by providing a quantitative determination of free MUC1fs in biological samples, for example blood plasma, using a method that is easy to perform, does not require spe-cialized instrumentation, is feasible in any biochemical laboratory, and allows to determine MUC1fs regardless of VNTR region length or the location of the mutation in a specific patient.

The determination of MUC1fs according to the invention proceeds as follows and each step is being followed by washing. The antibody against the mutated form of VNTR MUC1fs (anti-fs-VNTR-immob, amino acid sequence of a specific epitope that the antibody recognizes: SEQ ID NO. 1: SPRCHLGPGHQAGPGLHRPP) is immobilized on a support for example in a form of a gel or beads based on polymeric or magnetic material. The immobilized antibody is then incubated with a test sample of biological material, for example blood plasma, and the material is divided into two parts for the following procedure. To the first part, a detection monoclonal antibody against wild-type VNTR MUC1fs (anti-wt-VNTR, amino acid sequence of a specific epitope that the antibody recognizes: SEQ ID NO. 2: APDTRP) is added, followed by an antibody against anti-wt-VNTR (anti-anti-wt-VNTR) carrying horseradish peroxidase (HRP). To the second part, a detection monoclonal antibody against the mutated form of VNTR MUC1fs (anti-fs-VNTR, amino acid sequence of the specific epitope that the antibody recognizes: SEQ ID NO. 1: SPRCHLGPGHQAGPGLHRPP) is added, followed by antibody against anti-fs-VNTR (anti-anti-fs-VNTR) carrying HRP. In both cases, the quantification is then carried out by adding a suitable substrate, for example tetramethylbenzidine (TMB) and hydrogen peroxide, and the degree of color change is quantified spectrophotometrically. An alternative method is to use directly labeled detection antibodies or directly labeled antibodies against anti-wt-VNTR and anti-fs-VNTR carrying specific fluorophores or mass labels attached to their molecules and subsequent fluorimetric or mass-spectrometric quantification.

Results acquired by the abovementioned procedure are quantified using a calibration curve obtained using a specially developed standard based on bovine serum albumine (BSA) carrying antigenic epitopes of MUC1 and MUC1fs, specifically epitopes corre-sponding to wt-VNTR (amino acid sequence SEQ ID NO. 3: GVTSAPDTRPAPG), fs-VNTR (amino acid sequence SEQ ID NO. 1: SPRCHLGPGHQAGPGLHRPP), and a C-terminus specific for MUC1fs (amino acid sequence SEQ ID NO. 4: LSFYSGAQR). The concentration of anti-wt-VNTR and anti-fs-VNTR antibodies determined by quantification using the calibration curve corresponds to concentrations of non-mutated and mutated MUC1fs repeats in the examined biological sample.

Inventive and non-obvious part of the invention is the parallel use of two different antibodies, one against the wild-type VNTR MUC1fs and one against the mutated VNTR MUC1fs. After normalization and quantification of the results using the modified-BSA standard, it is not only possible to determine the concentration of MUC1fs in a biological sample in any variant, i.e. regardless of VNTR length or mutation position, but it is also possible to determine the VNTR length and mutation position in the variant present in a specific sample. In general, four types of results can be obtained:
1. Low concentration of anti-wt-VNTR and low concentration of anti-fs-VNTR
2. High concentration of anti-wt-VNTR and low concentration of anti-fs-VNTR
3. Low concentration of anti-wt-VNTR and high concentration of anti-fs-VNTR
4. High concentration of anti-wt-VNTR and high concentration of anti-fs-VNTR

The type 1 outcome represents samples from healthy individuals who do not have a mutation in the MUC1 gene. In exceptional cases, this may be a false negative result in patients with a very short VNTR region or a mutation very close to the C-terminus. In these cases, MUC1fs immobilization fails due to insufficient binding site for the immobilized antibody against the mutated VNTR MUC1fs. The type 2 outcome is observed in patients with a mutation near the C-terminus, the type 3 outcome in patients with a mutation near the N-terminus, and the type 4 outcome in patients with a mutation located in the middle of the VNTR region.

Free plasma MUC1fs is a suitable biomarker for differential diagnosis and monitoring of ADTKD-MUC1 disease progression. Although the disease manifests itself exclusively in the kidneys, the main producers of the mutated protein are large organs such as the lungs, intestines, spleen, or endocrine glands, which release it into the blood. Thus, the determination of MUC1fs in plasma is significantly more reliable than the determination, for example, in urinary tract cells. Furthermore, under normal circumstances in patients with ADTKD-MUC1, the plasma concentration of MUC1fs is relatively stable during their lifetime, so any observed change may indicate an emerging complication worthy of attention. This fact further allows the use of plasma MUC1fs concentration as a biomarker of the effectiveness of a used therapy. In the case of therapy based on blocking the MUC1 promoter, which inhibits the production of MUC1fs, or the use of drugs that promote the permeation of MUC1fs into lysosomes, and thus its metabolic degradation, the positive effect of the treatment is manifested by a reduction in plasma MUC1fs concentration. In contrast, in the case of therapies that promote the passage of MUC1fs through the cell wall into the extracellular space in order to relieve affected renal cells, in which MUC1fs accumulate pathologically, a successful treatment results in an increase in the plasma concentration of MUC1fs.

Testing for MUC1fs plasma levels is also important in individuals who do not have a history of ADTKD-MUC1 in their family. Because a significant number of tumors have a somatic mutation in the MUC1 gene, the presence of MUC1fs in the plasma of these patients is a biomarker that indicates a likely presence of tumor growth. Even in this case, MUC1fs can be used as a biomarker of disease progression, where increasing concentrations indicate deteriorating health state of the patient, or as a biomarker to monitor the effectiveness of a cytostatic therapy and disease relapse, where successful treatment and associated tumor regression will reduce MUC1fs concentration over time and relapse of the disease is indicated by increasing levels of the biomarker.

The present invention provides a tool for monitoring the values or changes in the values of said biomarker in the form of MUC1fs, thus enabling monitoring of the progression of the disease or the effectiveness of its therapy.

### BRIEF DESCRIPTION OF DRAWINGS

FIG. 1

FIG. 1 schematically shows a system for determining the plasma concentration of MUC1fs. The left diagram shows the determination of the length of fs-VNTR, the right diagram shows the determination of the length of wt-VNTR. The “+C” mark indicates the position of the mutation.

FIG. 2

FIG. 2 schematically shows a standard based on modified BSA. The letters K represent chains of lysine amino acid.

FIG. 3

FIG. 3 schematically shows the relationship between the measured normalized anti-wt-VNTR and anti-fs-VNTR concentrations, the length of the VNTR region, and the mutation position.

FIG. 4

FIG. 4 shows a graph of measured anti-wt-VNTR and anti-fs-VNTR concentrations in a control group of healthy individuals. Each point represents one person.

FIG. 5

FIG. 5 shows a graph of measured anti-wt-VNTR and anti-fs-VNTR concentrations in a group of patients with ADTKD-MUC1. Each point represents one person.

FIG. 6

FIG. 6 shows a graph of measured anti-wt-VNTR and anti-fs-VNTR concentrations in a group of individuals unaffected by ADTKD-MUC1. Each point represents one person. The twelve individuals showing the presence of various forms of MUC1fs were subsequently diagnosed with cancer.

FIG. 7

FIG. 7 shows graphs of measured anti-wt-VNTR and anti-fs-VNTR concentrations plotted against time in a patient with ADTKD-MUC1 undergoing an experimental treatment with MUC1 promoter blockade. Thus, it illustrates the ability of the invention to monitor the development of plasma concentration of MUC1fs during a therapy.

FIG. 8

FIG. 8 shows graphs of measured anti-wt-VNTR and anti-fs-VNTR concentrations plotted against time in a patient with ADTKD-MUC1 undergoing an experimental treatment with a substance that promotes MUC1fs permeation into lysosomes. Thus, it illustrates the ability of the invention to monitor the development of plasma concentration of MUC1fs during a therapy.

FIG. 9

FIG. 9 shows graphs of measured anti-wt-VNTR and anti-fs-VNTR concentrations plotted against time in a patient with ADTKD-MUC1 undergoing an experimental treatment with a substance that promotes MUC1fs permeation through the cell membrane into the extracellular space. Thus, it illustrates the ability of the invention to monitor the development of plasma concentration of MUC1fs during a therapy.

FIG. 10

FIG. 10 shows graphs of measured anti-wt-VNTR and anti-fs-VNTR concentrations plotted against time in a patient with ovarian cancer and undergoing cytostatic treatment. Thus, it illustrates the ability of the invention to monitor the development of plasma concentration of MUC1fs during a therapy.

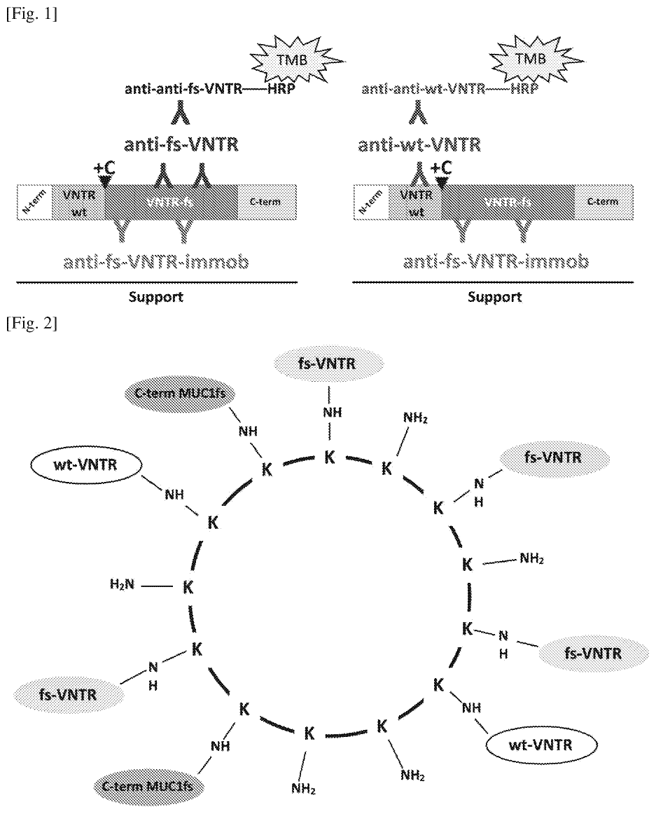

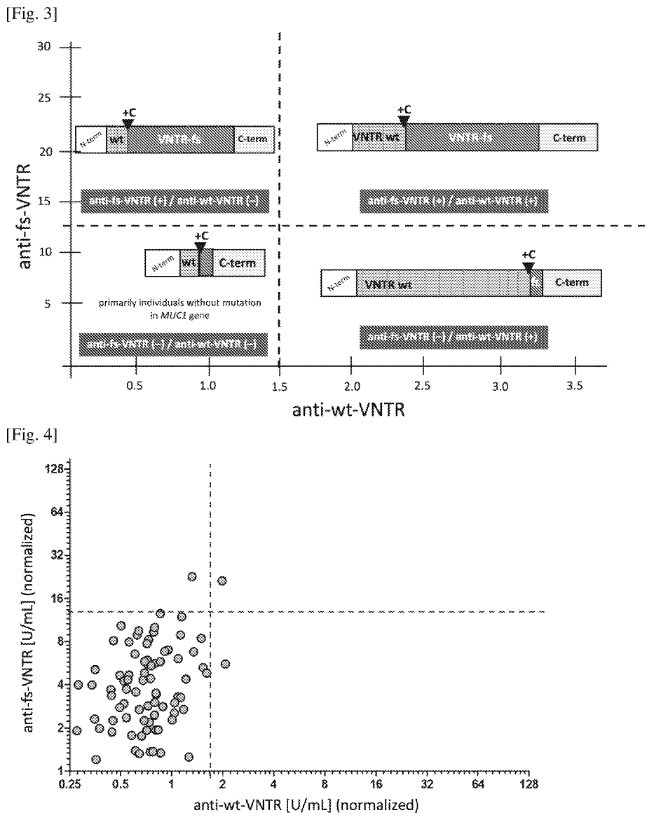

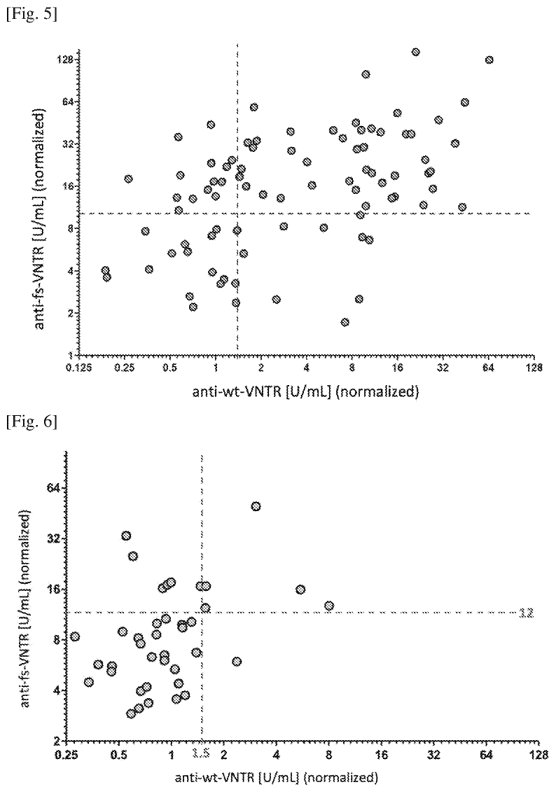

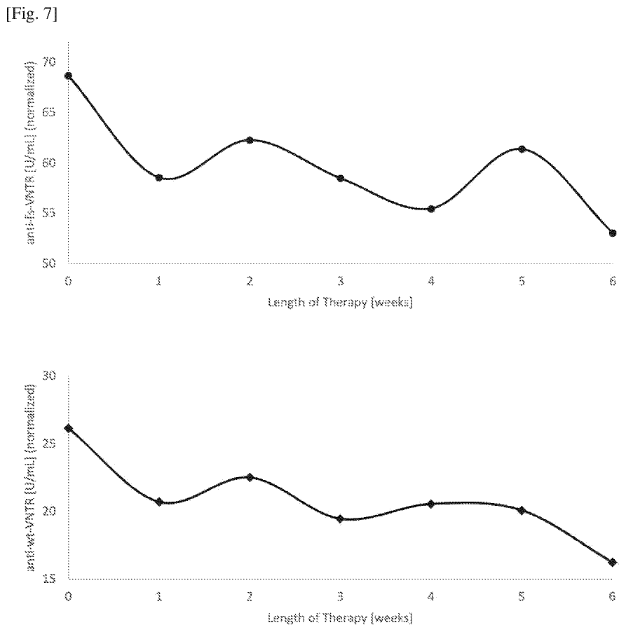

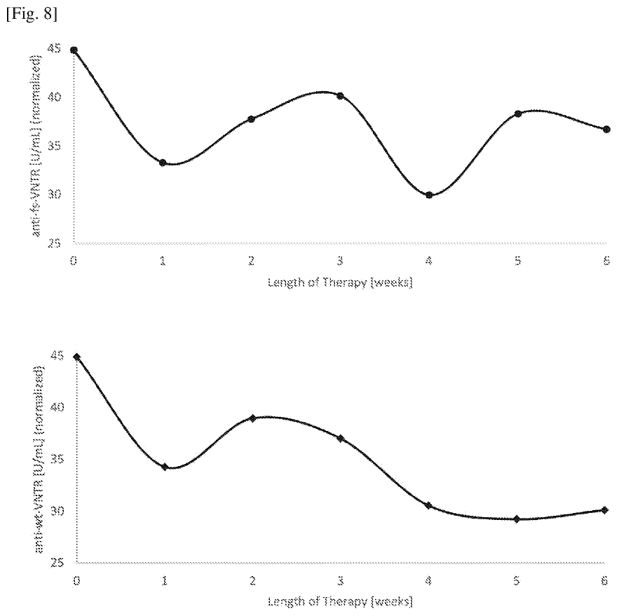

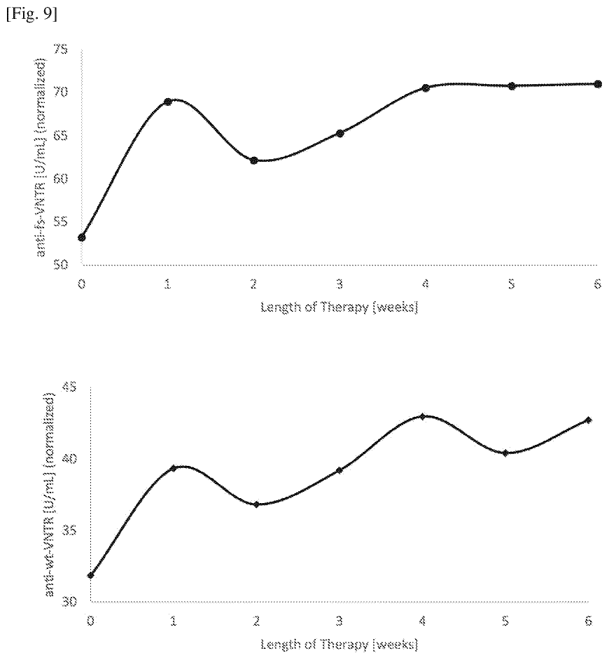

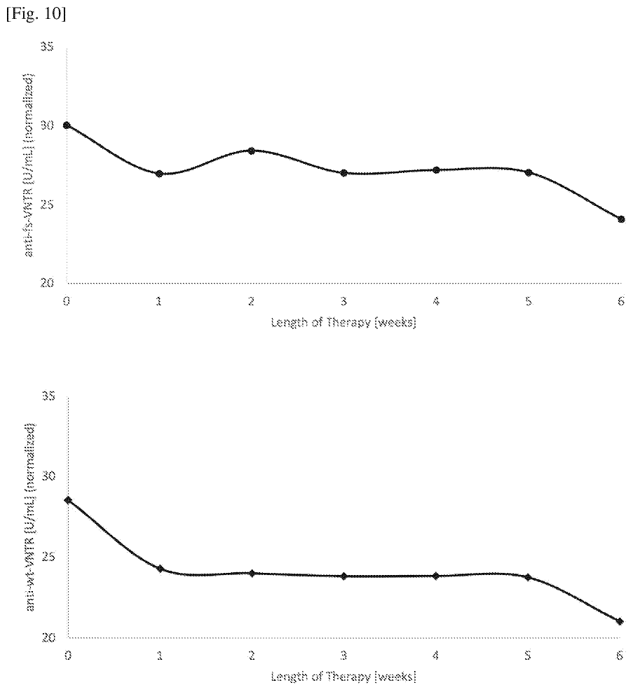

### DESCRIPTION OF EMBODIMENTS

### Example 1

Example 1 describes the determination of the concentration of MUC1fs in a blood plasma sample using a method performed on a system according to the invention.

Into wells of two microtiter plates containing antibody-immobilization resin, 100 μL of phosphate buffer solution with 0.15 M NaCl (Phosphate Buffered Saline, PBS) containing antibody against the mutated form of VNTR MUC1fs (amino acid sequence of the specific epitope that the antibody recognizes: SEQ ID NO. 1: SPRCHLGPGHQAGPGLHRPP) at a concentration of 5 μg/mL is added and the plates are incubated overnight at 4° C. After aspirating and washing the wells three times with PBS washing solution containing 0.05% of polysorbate 20, the wells are filled with blocking solution, i.e. 1% solution of unmodified BSA in PBS containing 0.05% of polysorbate 20, and the plates are left for one hour at laboratory temperature. After aspirating the blocking solution, the plate is washed once with the washing solution. A standard in a serial dilution (2×) is added to a portion of the wells of each plate, starting at 1:32 for anti-wt-VNTR MUC1fs antibody detection (Plate 1) and 1:256 for anti-fs-VNTR MUC1fs antibody detection (Plate 2). A mixture containing a blood plasma sample obtained by centrifugation of collected peripheral venous blood twice diluted with PBS, 1% of unmodified BSA, and 0.05% of polysorbate 20 is added to the other wells and the plates are incubated for two hours at laboratory temperature. After that, the plates are aspirated and washed with the washing solution three times. Mouse monoclonal antibody against the wild-type of VNTR MUC1fs (amino acid sequence of the specific epitope that the antibody recognizes: SEQ ID NO. 2: APDTRP) at a dilution of 1:250 is added to Plate 1. After 2 hours of incubation at laboratory temperature, the plate is washed three times with the washing solution and an antibody against the mouse antibody carrying HRP at a dilution of 1:5,000 is added. The plate is incubated with this antibody for 1 hour at laboratory temperature. Rabbit antibody against the mutated form of VNTR MUC1fs (amino acid sequence of the specific epitope that the antibody recognizes: SEQ ID NO. 1: SPRCHLGPGHQAGPGLHRPP) at a dilution of 1:1,000 is added to Plate 2. After 2 hours of incubation at laboratory temperature, the plate is washed three times with the washing solution and an antibody against the rabbit antibody carrying HRP at a dilution of 1:5,000 is added. The plate is incubated with this antibody for 1 hour at laboratory temperature. Subsequently, both plates are aspirated and washed three times with PBS solution. Then, 100 μL of a standard solution containing tetramethylbenzidine (TMB) and hydrogen peroxide is added to both plates. The color-change reaction is terminated after 15 minutes by an addition of 1 M sulfuric acid. Absorbance of the solutions in both individual plates is measured spectrophotometrically at 450 nm. Alternatively, directly labeled detection antibodies or directly labeled antibodies against anti-wt-VNTR and anti-fs-VNTR antibodies carrying specific fluorophores or mass labels attached to their molecules are used and quantified fluorimetrically or by mass spectrometry. To determine the concentration of MUC1fs in the plasma sample, a calibration curve is further prepared according to the abovementioned procedure using a modified-BSA standard at various dilutions. A 16-fold dilution represents 100 U/mL of repeats of the wild-type VNTR MUC1fs and a 256-fold dilution represents 100 U/mL of repeats of the mutated VNTR MUC1fs. The measured concentrations of the real samples are normalized to a normalization sample with the determined concentrations of 40 U/mL of anti-fs-VNTR and 6 U/mL of anti-wt-VNTR.

### Example 2

Example 2 demonstrates monitoring of the efficacy of an experimental treatment by blocking the MUC1 promoter in a patient suffering from ADTKD-MUC1 using the method of the invention.

A peripheral venous blood sample, from which plasma is separated by centrifugation, is taken from the patient at weekly intervals for six weeks. The presence of MUC1fs in plasma is then determined by the method described in Example 1. The measured normalized values of anti-fs-VNTR and anti-wt-VNTR concentrations and their development over time are summarized in the following table:

| Length of Therapy | anti-fs-VNTR [U/mL] | anti-wt-VNTR [U/mL] |
|---|---|---|
| beginning | 68.678 | 26.151 |
| 1 week | 58.537 | 20.707 |
| 2 weeks | 62.265 | 22.514 |
| 3 weeks | 58.472 | 19.460 |
| 4 weeks | 55.458 | 20.559 |
| 5 weeks | 61.368 | 20.089 |
| 6 weeks | 53.037 | 16.252 |

### Example 3

Example 3 demonstrates monitoring of the efficacy of an experimental treatment with a substance that promotes a permeation of MUC1fs into lysosomes in a patient suffering from ADTKD-MUC1 using the method of the invention.

A peripheral venous blood sample, from which plasma is separated by centrifugation, is taken from the patient at weekly intervals for six weeks. The presence of MUC1fs in plasma is then determined by the method described in Example 1. The measured normalized values of anti-fs-VNTR and anti-wt-VNTR concentrations and their development over time are summarized in the following table:

| Length of Therapy | anti-fs-VNTR [U/mL] | anti-wt-VNTR [U/mL] |
|---|---|---|
| beginning | 44.828 | 44.885 |
| 1 week | 33.292 | 34.268 |
| 2 weeks | 37.762 | 38.967 |
| 3 weeks | 40.168 | 37.007 |
| 4 weeks | 29.970 | 30.542 |
| 5 weeks | 38.300 | 29.224 |
| 6 weeks | 36.724 | 30.101 |

### Example 4

Example 4 demonstrates monitoring of the efficacy of an experimental treatment with a substance that increases a permeation of MUC1fs through the cell membrane into the extracellular space in a patient suffering from ADTKD-MUC1 using the method of the invention.

A peripheral venous blood sample, from which plasma is separated by centrifugation, is taken from the patient at weekly intervals for six weeks. The presence of MUC1fs in plasma is then determined by the method described in Example 1. The measured normalized values of anti-fs-VNTR and anti-wt-VNTR concentrations and their development over time are summarized in the following table:

| Length of Therapy | anti-fs-VNTR [U/mL] | anti-wt-VNTR [U/mL] |
|---|---|---|
| beginning | 53.235 | 31.845 |
| 1 week | 68.964 | 39.332 |
| 2 weeks | 62.187 | 36.815 |
| 3 weeks | 65.315 | 39.201 |
| 4 weeks | 70.552 | 42.963 |
| 5 weeks | 70.785 | 40.405 |
| 6 weeks | 71.025 | 42.712 |

### Example 5

Example 5 demonstrates monitoring of the efficacy of a cytostatic treatment in a patient suffering from ovarian cancer using the method of the invention.

A peripheral venous blood sample, from which plasma is separated by centrifugation, is taken from the patient at weekly intervals for six weeks. The presence of MUC1fs in plasma is then determined by the method described in Example 1. The measured normalized values of anti-fs-VNTR and anti-wt-VNTR concentrations and their development over time are summarized in the following table:

| Length of Therapy | anti-fs-VNTR [U/mL] | anti-wt-VNTR [U/mL] |
|---|---|---|
| beginning | 30.036 | 28.552 |
| 1 week | 26.966 | 24.295 |
| 2 weeks | 28.413 | 24.011 |
| 3 weeks | 26.997 | 23.829 |
| 4 weeks | 27.183 | 23.840 |
| 5 weeks | 27.028 | 23.761 |
| 6 weeks | 24.074 | 21.036 |

### INDUSTRIAL APPLICABILITY

Method for determination of mutated form of mucin-1 protein in a biological sample is industrially applicable in diagnostics of chronic hereditary kidney disease and cancer using laboratory analysis of clinical samples.

1. Method for determination of concentration of mutated form of mucin-1 protein in a biological sample and determination of position of its mutation, characterized in that it comprises steps of adding a biological sample to an immobilized antibody against the mutated form of variable number tandem repeats (VNTR) of mutated frame-shifted variant of mucin-1 protein (MUC1fs), adding an antibody against the wild-type form of VNTR MUC1fs to a portion of this mixture and performing a quantitative analysis, and adding an antibody against the mutated form of VNTR MUC1fs to the remainder of the mixture of the biological sample with the immobilized antibody against the mutated form of VNTR MUC1fs and performing quantitative analysis.

2. Method according to claim 1, characterized in that the amino acid sequence of a specific epitope that the antibody against the mutated form of VNTR MUC1fs recognizes is SEQ ID NO. 1.

3. Method according to claim 1, characterized in that the amino acid sequence of a specific epitope that the antibody against the wild-type form of VNTR MUC1fs recognizes is SEQ ID NO. 2.

4. Method according to claim 1, characterized in that the quantitative analysis is performed using an antibody against the wild-type form of VNTR MUC1fs carrying a structure selected from the group consisting of horseradish peroxidase (HRP) enzyme with subsequent addition of a mixture of reactive dye and hydrogen peroxide and measurement of a change in absorbance of the solution or fluorescent label with a subsequent measurement of a change in fluorescence value or mass label with a subsequent measurement of a change in mass by mass spectrometry.

5. Method according to claim 1, characterized in that the quantitative analysis is performed using an antibody against the mutated form of VNTR MUC1fs carrying a structure selected from the group consisting of HRP enzyme with subsequent addition of a mixture of reactive dye and hydrogen peroxide and measurement of a change in absorbance of the solution or fluorescent label with a subsequent measurement of a change in fluorescence value or mass label with a subsequent measurement of a change in mass by mass spectrometry.

6. Method according to claim 1, characterized in that the quantitative analysis is performed using an antibody against the antibody against the wild-type form of VNTR MUC1fs, while the antibody against the antibody is carrying a structure selected from the group consisting of HRP enzyme with subsequent addition of a mixture of reactive dye and hydrogen peroxide and measurement of a change in absorbance of the solution or fluorescent label with a subsequent measurement of a change in fluorescence value or mass label with a subsequent measurement of a change in mass by mass spectrometry.

7. Method according to claim 1, characterized in that the quantitative analysis is performed using an antibody against the antibody against the mutated form of VNTR MUC1fs, while the antibody against the antibody is carrying a structure selected from the group consisting of HRP enzyme with subsequent addition of a mixture of reactive dye and hydrogen peroxide and measurement of a change in absorbance of the solution or fluorescent label with a subsequent measurement of a change in fluorescence value or mass label with a subsequent measurement of a change in mass by mass spectrometry.

8. Method according to claim 1, characterized in that for determination of the concentration of MUC1fs in the sample, determination of the VNTR length, and determination of the mutation position, a length of the mutated region of VNTR MUC1fs in the sample is determined using an appropriate calibration curve obtained by analyzing a standard containing at least one epitope of the mutated form of VNTR MUC1fs and a length of the wild-type region of VNTR MUC1fs in the sample is determined using an appropriate calibration curve obtained by analyzing a standard containing at least one epitope of the wild-type form of VNTR MUC1fs.

9. (canceled)
